(19) United States
(12) Patent Application Publication
(10) Pub. No.: US 2024/0366757 A1
(43) Pub. Date: Nov. 7, 2024

(54) FORMULATIONS OF IMMUNE CHECK POINT INHIBITORS OR LIKE

(71) Applicant: DR. REDDY’S LABORATORIES LIMITED, Hyderabad (IN)

(72) Inventors: Murali JAYARAMAN, Kancheepuram (Distt) (IN); Saisharan K GOUD, Kamareddy (Distt) (IN); Sunil Ashok NANKAR, Jalgaon (Distt) (IN); Indra Kumar SIGIREDDI, Kharagpur (IN); Suman LABALA, Srikakulam (IN); Giridhar Purushottam TALABATTULA, Rayapuram Samalkot (IN); Lalithkumar BASKARAN, Vellore (IN); Shalaka R, Chennai (IN)

(21) Appl. No.: 18/689,476

(22) PCT Filed: Sep. 7, 2022
(86) PCT No.: PCT/IN2022/050802

§ 371 Date: Mar. 6, 2024

(30) Foreign Application Priority Data
Sep. 7, 2021 (IN) 202141040524

(51) Int. Cl. A61K 39/395; A61K 9/08; A61K 47/26; C07K 16/28

(52) U.S. Cl. CPC A61K 39/39591; A61K 9/08; A61K 47/26; C07K 16/2818

(57) ABSTRACT

The present invention discloses a stable buffer free formulation of anti-PD1/anti-PD-L1 antibody, comprising an anti-PD1 or an anti-PD-L1 antibody, water, mannitol and surfactant, and stabilized at a pH of about 5.0-about 6.0. The disclosed antibody formulation is a liquid formulation and can be lyophilized. Further, the said formulation is also suitable for different mode of administration such as subcutaneous/intravenous, for therapeutic use.

### FIELD OF THE INVENTION

The present invention is related to an aqueous, buffer free formulation of an antibody molecule, stabilized at a particular pH, without any buffering agent. The disclosed formulation stabilizes the antibody from about 10 mg/ml to about 200 mg/ml which are suitable for intravenous or subcutaneous route of administration.

### BACKGROUND

Over the past two decades, recombinant DNA technology has led to the commercialization of many proteins, particularly antibody therapeutics. The effectiveness of these therapeutic antibodies is majorly dependent on the stability, route of administration and their dosage forms and concentrations. This in turn, necessitates therapeutic antibodies to be formulated appropriately to retain the stability and activity of a therapeutic antibody.

Formulations for each route of administration and dosage forms may be unique and, therefore, have specific requirements. Solid dosage forms, such as lyophilized powders, are generally more stable than liquid (aqueous) formulations. However, reconstitution of the lyophilized formulation requires a significant vial overfill, care in handling and involves high production cost relative to a liquid formulation. While liquid formulations are advantageous in these and are usually preferred for injectable protein therapeutics (in terms of convenience for the end user and ease of preparation for the manufacturer), this form may not always be feasible given the susceptibility of proteins to denaturation, aggregation and oxidation under stresses such as temperature, pH changes, agitation etc. All of these stress factors could result in the loss of biological activity of a therapeutic protein/antibody.

In particular, high concentration liquid formulations are susceptible to degradation and/or aggregation. Nevertheless, high concentration formulations may be desirable for subcutaneous or intravenous route of administration, as the frequency of administration and injection volume is reduced. On the other hand, specific treatment schedule and dosing might require a low concentration formulation and prefer intravenous route of administration for more predictable delivery and complete bioavailability of the therapeutic drug.

A formulation combination with increased concentration of protein and/or stabilizers may increase the viscosity of the formulation, in turn increasing the injection time and pain at the site of injection and also pose difficulties during processing of the drug substance. Hence, it is necessary to develop an improved formulation, which stabilizes a protein at a wide range of its concentration and suitable for in different route of administration (intravenous or subcutaneous), pose a significant developmental challenge. Further, every protein or antibody with its unique characteristics and properties of degradation, adds to the complexity in the development of a stable formulation and may demand a specific formulation.

### SUMMARY

The present invention discloses a buffer free formulation of an anti-PD1 or an anti-PDL1 antibody comprising, an anti-PD1/PD-L1 antibody, water and surfactant, wherein the antibody concentration is 10 mg/ml to 200 mg/ml. The antibody formulated in water maintains solubility and stability, during long-term liquid storage or other processing steps, such as freeze/thawing.

In particular, the invention discloses a buffer free formulation of an anti-PD1 or an anti-PD-L1 antibody, comprising an anti-PD1 or an anti-PD-L1 antibody, mannitol or sorbitol or trehalose, water and surfactant, stabilized at a pH of about 5.0 to about 6.0. The disclosed anti-PD1 or anti-PD-L1 antibody formulation of the invention does not require any specific buffering agent to maintain/stabilize the pH of the formulation.

In another aspect, the invention discloses a method of stabilizing an anti-PD1 or an anti-PD-L1 antibody in an aqueous solution, at a pH of about 5.0 to about 6.0, without a buffering agent comprising steps of; expressing and purifying anti-PD1 or anti-PDL1 antibody to obtain anti-PD1 or anti-PDL1 antibody composition, subjecting the said antibody composition to diafiltration with a diafiltration media comprising water, followed by addition of one or more pharmaceutically acceptable excipients. The pharmaceutically acceptable excipients is mannitol or trehalose or sorbitol, salt, amino acid or surfactant.

The formulations and methods disclosed in the invention stabilizes anti-PD1 or anti-PD-L1 antibody, in concentrations ranging from about 10 mg/ml to about 200 mg/ml.

In yet another aspect, the disclosed buffer free anti-PD1 or anti-PD-L1 antibody formulation exhibits colloidal stability. And the viscosity of the disclosed anti-PD1 or anti-PDL1 formulations is less than 20 cP.

The disclosed formulations of the invention, specifically controls/resists a change in the basic variants content of the antibody over a period of time. This is particularly advantageous since charge variants (including basic variants) content is a critical quality attribute and any change in the content may influence the stability of the antibody molecule.

The disclosed formulations of the antibody exhibits stability under accelerated conditions such as at 40° C. for at least two weeks or at 25° C. for four weeks.

### DETAILED DESCRIPTION OF THE INVENTION

### Definitions

The term “about” used herein would mean and include a variation of up to 20% from the particular value.

The term “antibody” as used herein encompasses whole antibodies or an antigen binding fragment (i.e., “antigen-binding portion”) or fusion protein thereof.

The term “buffer or buffering agent” used interchangeably herein this document, refers to an agent which resists any change in pH of a solution, near a chosen value, up on addition of acid or base.

The term “stable” formulation refers to the formulation wherein the antibody therein retains its physical stability and/or chemical stability and/or biological activity.

Stability studies provides evidence of the quality of an antibody under the influence of various environmental factors during the course of time. ICH's “Q1A: Stability Testing of New Drug Substances and Products,” states that data from accelerated stability studies can be used to evaluate the effect of short-term excursions higher or lower than label storage conditions that may occur during the shipping of the antibodies.

Various analytical methods are available for measuring the physical and chemical degradation of the antibody in the pharmaceutical formulations. An antibody “retains its physical stability” in a pharmaceutical formulation if it shows substantially no or minimal (or to the extent of acceptable standards) signs of aggregation, precipitation and/or denaturation upon visual examination of color and/or clarity, or as measured by UV light scattering or by size exclusion chromatography. An antibody is said to “retain its chemical stability” in a pharmaceutical formulation when its shows no or minimal formation of product variants which may include variants as a result of chemical modification of antibody of interest such as deamination, oxidation etc. Analytical methods such as ion exchange chromatography and hydrophobic ion chromatography may be used to investigate the chemical product variants.

The term ‘monomer’ as used herein describes antibodies consisting of two light chains and two heavy chains. The monomer content of an antibody composition is typically analyzed by size exclusion chromatography (SEC). As per the separation principle of SEC the large molecules or molecules with high molecular weight (HMW) elute first followed by smaller or lower weight molecules. In a typical SEC profile for an antibody composition, aggregates that may include dimers, multimers, etc., elute first, followed by monomer, and the clipped antibody variants or degradants may be eluted last. In some circumstances the aggregate peak or the degradant peaks may not elute as a baseline separated peaks but instead as a shoulder or abnormal broad peaks. In order to maintain the appropriate activity of an antibody, in particular of a therapeutic antibody, it is desirable to reduce the formation of aggregate or fragmentation of products and hence control the monomer content to a target value. Ability to inhibit the formation of aggregate and degradant content as measured at various time points during stability studies may indicate the suitability of the candidate formulation for antibody of interest. TSK-GEL G3000SWXL (7.8 mm×30 cm) column from TOSCH can be used on water HPLC to perform SEC.

The term ‘main peak’ as used herein refers to the peak that elutes in abundance (major peak) during a cation exchange chromatography. The peak that elutes earlier than the main peak, during a cation exchange chromatography, with a charge that is acidic relative to the main peak is termed acidic variant peak. The peak that elutes later than the main peak, during a cation exchange chromatography, with a charge that is relatively basic than the main peak is termed as basic variant peak. The main peak content can be determined by Ion exchange chromatography (IEC). There are two modes of IEC available viz., cation and anion exchange chromatography. Negatively charged molecules bind to anion exchange resins while positively charged molecules bind to cation exchange resins. In a typical cation exchange chromatographic profile of an antibody composition acidic variants elute first followed by the main peak and thereafter lastly the basic variants will be eluted. The acidic variants are a result of antibody modifications such as deamidation of asparagine residues. The basic variants are a result of incomplete removal of C-terminal lysine residue(s). In general, in an antibody a lysine residue is present at the C-terminal end of both heavy and light chain. An antibody molecule containing lysine at both heavy and light chain is referred to as K2 variant, the antibody molecule containing lysine residue at either one of heavy and light chain is referred to as K1 variant and antibody molecule having none is K0 molecule. Carboxypeptidase B (CP-B enzyme) enzyme acts on the C-terminal lysine residues present on K2 and K1 variants and thus converting them as K0 molecules. As per circumstances of the case, the IEC analysis can be carried out for samples digested with carboxypeptidase B (CP-B) enzyme. In a typical stability study it is expected that a stable formulation leads to reduction in formation of charge variants (acidic and basic variants), during the study, and hence minimize any reduction in main peak content.

Pharmaceutically acceptable excipients refer to the additives or carriers, which may contribute to stability of the antibody in formulation. The excipients may encompass stabilizers and tonicity modifiers. Examples of stabilizers and tonicity modifiers include, but not limited to, salts, surfactants, and derivatives and combination thereof.

The term sugar/s as used herein includes organic compounds having general formula of all carbohydrates of the general formula Cn(H2O)n. Sugars can be referred to monosaccharides, disaccharides, and polysaccharides. Examples of sugars include, but are not limited to, sucrose, trehalose, glucose, dextrose, raffinose and others.

The term “polyol” or “sugar alcohol” as used herein includes an organic compound containing multiple hydroxyl groups. Examples of polyols include mannitol, sorbitol, xylitol etc.

Surfactant refers to pharmaceutically acceptable excipients used to protect the protein formulations against various stress conditions, like agitation, shearing, exposure to high temperature etc. The suitable surfactants include but are not limited to polyoxyethylensorbitan fatty acid esters such as Tween 20™ or Tween 80™, polyoxyethylene-polyoxypropylene copolymer (e.g. Poloxamer, Pluronic), sodium dodecyl sulphate (SDS) and the like or combination thereof.

The term “fragments” herein refers to a part of large entity such as part of protein or antibody which consists of less than the entire amino acid sequence of the protein or the antibody which are formed due to terminal or internal deletion of a portion of the protein/antibody.

The term “charge variants” herein refers to an antibody variants which has net positive or negative charge and contains either lower or higher isoelectric point (pI) than the antibody of interest. Examples of charge variants include acidic variants and basic variants. The acidic variants of an antibody can be formed due to deamidation of glutamine and aspargine and sialylation which may impart net negative charge to the antibody and resulted in decrease in pI of the antibody. The basic variants of an antibody can be formed due to C-terminal lysine variation, oxidation, glycine amidation, succinamide formation, removal of sialic acids which may impart net positive charge to the antibody and resulted in increase in pI of the antibody.

### DETAILED DESCRIPTION OF THE EMBODIMENTS

The present invention discloses a buffer free formulation of an anti-PD1 or anti-PD-L1 antibody, comprising anti-PD1 or/anti-PD-L1 antibody, water and surfactant, stabilized at a pH of about 5.0 to about 6.0. Wherein the formulation is devoid of any buffering agent.

In one embodiment, the invention discloses a buffer free formulation of an anti-PD1 or an anti-PD-L1 antibody, stabilized at a pH of about 5.0-about 6.0, comprising anti-PD1 or anti-PD-L1 antibody, water and surfactant, wherein the pH of the anti-PD1 or anti-PD-L1 antibody formulation is maintained without any buffering agent.

In the above mentioned embodiment, the anti-PD1 or anti-PD-L1 antibody formulation optionally contains one or more pharmaceutically acceptable excipients and the one or more pharmaceutically acceptable excipients comprise sugar or polyol or salt or amino acid or surfactant.

In an embodiment, the invention discloses a buffer free formulation of an anti-PD1 or an anti-PD-L1 antibody, stabilized at a pH of about 5.0-about 6.0, comprising anti-PD1 or anti-PD-L1 antibody, water, mannitol or sorbitol or trehalose, and surfactant, wherein the antibody concentration is from 10 mg/ml to 200 mg/ml and pH of the formulation is maintained without any buffering agent.

In the above mentioned embodiment, the anti-PD1 or anti-PD-L1 antibody optionally contains amino acid and/or salts.

In another embodiment, the invention discloses a method of stabilizing an anti-PD1 or an anti-PD-L1 antibody in a solution, at a pH of about 5.0 to about 6.0 without a buffering agent, the method comprising;
a) expressing and purifying an anti-PD1 or an anti-PDL1 antibody to obtain the antibody composition;
b) subjecting the purified antibody composition to a diafiltration step using water as a diafiltration medium,
c) obtaining the antibody composition in water,
d) addition of polyol and/or salt to the antibody composition obtained from step c) to obtain anti-PD1 or anti-PDL1 antibody solution; and
   wherein the anti-PD1 or anti-PD-L1 antibody solution obtained by the said method exhibits stability at 40° C. for 2 weeks.

In the above mentioned embodiment, the antibody composition obtained in step c) may optionally be subjected for ultrafiltration to concentrate up to 200 mg/ml.

In an embodiment, the invention discloses a method of controlling fragmentation of an anti-PD1 or an anti-PD-L1 antibody in an aqueous buffer free formulation, stabilized at a pH of about 5.0 to about 6.0, wherein the method comprises formulating the anti-PD1 or anti-PDL1 antibody in a composition comprising water, mannitol salt and surfactant, and wherein the pH of the formulation is maintained without any buffering agent. The obtained anti-PD1 or anti-PD-L1 antibody formulation from the said method is stable at 40° C. for two weeks and content of the said antibody fragmentation is less than 1% after storage at 40° C. for two weeks.

In an embodiment, the invention discloses a method of controlling aggregation in an anti-PD1 or an anti-PD-L1 antibody in an aqueous buffer free formulation, stabilized at a pH of about 5.0 to about 6.0, wherein the method comprises formulating the anti-PD1 or anti-PD-L1 antibody in a composition comprising water, mannitol, salt and surfactant, and wherein the pH of the formulation is maintained without any buffering agent. The obtained anti-PD1 or anti-PD-L1 antibody formulation from the said method is stable at 40° C. for two weeks and aggregate content of the said antibody formulation is less than 1.5% after storage at 40° C. for two weeks.

In an embodiment, the invention discloses a method of controlling formation of charge variants in an anti-PD1 or an anti-PD-L1 antibody in an aqueous buffer free formulation, stabilized at a pH of about 5.0 to about 6.0, wherein the method comprises formulating the anti-PD1 or anti-PD-L1 antibody in a composition comprising water, mannitol, salt and surfactant, and wherein the pH of the formulation is maintained without any buffering agent. The obtained anti-PD1 or anti-PD-L1 antibody formulation from the said method is stable at 40° C. for two weeks and change in charge variants content of the antibody is less than 10% when stored at 40° C. for two weeks.

In an embodiment, the invention discloses a method of controlling formation of basic variants in an anti-PD1 or an anti-PD-L1 antibody in an aqueous buffer free formulation, stabilized at a pH of about 5.0 to about 6.0, wherein the method comprises formulating the anti-PD1 or anti-PD-L1 antibody in a composition comprising water, mannitol, salt and surfactant, and wherein the pH of the formulation is maintained without any buffering agent. The obtained anti-PD1 or anti-PD-L1 antibody formulation from the said method is stable at 40° C. for two weeks and change in basic variants content of the antibody is less than 1% when stored at 40° C. for two weeks.

In another embodiment, the invention discloses a method of controlling change in basic variants content of an anti-PD1 or anti-PDL1 antibody, in an anti-PD1 or an anti-PD-L1 antibody composition, the method comprises formulating the anti-PD1 or anti-PD-L1 antibody in a composition comprising water, wherein the pH of the formulation is maintained at a pH of about 5.0 to about 6.0, without any buffering agent. The obtained anti-PD1 or anti-PD-L1 antibody formulation from the said method is stable at 40° C. for one to two weeks and change in basic variants content of the antibody is less than 1%.

In the above embodiment, the change in basic variants content is less than 1%, when stored at 40° C. for one or two weeks.

In the above mentioned embodiments, the formulation may additionally comprise sugar or polyol or salt or amino acid or surfactant.

In an embodiment, the invention discloses a method of controlling change in basic variants content of an anti-PD1 or anti-PDL1 antibody, in an anti-PD1 or an anti-PD-L1 antibody composition, the method comprises formulating the antibody in buffer free composition, wherein the change in basic variants of the antibody composition is less than 1% when the antibody composition is stored at 40° C. for one or two weeks as compared to the same antibody composition formulated in buffer wherein the change in basic variants is at least 2 to 5%.

In the above said embodiments, the concentration of anti-PD1/anti-PD-L1 antibody is 10 mg/ml or 20 mg/ml or 30 mg/ml or 40 mg/ml or 50 mg/ml, ‘or’ 60 mg/ml, ‘or’ 70 mg/ml, ‘or’ 80 mg/ml, ‘or’ 90 mg/ml, ‘or’ 100 mg/ml, ‘or’ 110 mg/ml, ‘or’ 120 mg/ml, ‘or’ 130 mg/ml, ‘or’ 140 mg/ml, ‘or’ 150 mg/ml, ‘or’ 160 mg/ml, ‘or’ 170 mg/ml, ‘or’ 180 mg/ml, ‘or’ 190 mg/ml, ‘or’ 200 mg/ml.

In any of the above mentioned embodiments, the claimed formulations of the invention exhibit stability under at least one of the following conditions, wherein the temperature range from 25° C. to 50° C. for a period of time which includes from 1 day to 6 months.

In any of the above said embodiment, the buffer free anti-PD1 or anti-PD-L1 antibody formulation exhibits stability at room temperature for at least 3 days, at least 7 days or at least 14 days or at least 28 days.

In an embodiment, the invention discloses a buffer free formulation of an anti-PD1 or anti-PDL1 antibody, stabilized at a pH of about 5.0 to about 6.0, comprising at least 10 mg/ml of anti-PD1 or anti-PDL1 antibody, water, mannitol, salt and surfactant, wherein the formulation is stable for at least two weeks when stored at 40° C.

In the above said embodiment, the anti-PD1 or anti-PD-L1 antibody formulation is stable and maintains at least 98% of monomeric content of the antibody, when stored at 40° C. for two weeks.

In an embodiment, the invention discloses a buffer free formulation of an anti-PD1/anti-PD-L1 antibody, stabilized at a pH of about 5.0 to about 6.0, comprising at least 10 mg/ml of anti-PD1 or anti-PD-L1 antibody, water, mannitol, salt and surfactant, wherein the formulation is stable for two weeks when stored at 40° C. and aggregate content of the antibody is less than 1.5% after storage at 40° C. for two weeks.

The anti-PD1 or anti-PDL1 antibody formulations disclosed in the invention are biologically active.

In any of the above mentioned embodiments, the pH of the disclosed formulation of the present invention is in the range from about 5.0 to about 6.0.

In any of the above mentioned embodiments, the pH of the disclosed formulation of the present invention is 5.5±0.5.

In an embodiment, the invention discloses, a buffer free nivolumab antibody formulation having pH of 5.0 to 6.0 comprising nivolumab antibody, water, mannitol, salt and surfactant, wherein the antibody concentration is from 10 mg/ml to 200 mg/ml and pH of the formulation is maintained without any buffering agent.

In another embodiment of the invention disclose a buffer free pembrolizumab antibody formulation having pH of 5.0 to 6.0 comprising pembrolizumab antibody, water, trehalose or sorbitol and surfactant, wherein the antibody concentration is from 10 mg/ml to 200 mg/ml and pH of the formulation is maintained without any buffering agent.

In any of the above embodiments of the invention, the stable liquid/aqueous formulation is suitable and can be lyophilized as lyophilized powders. Further, the lyophilized formulation of anti-PD1 or anti-PDL1 antibody can be reconstituted with appropriate diluent to achieve the liquid formulation suitable for administration.

In any of the above mentioned embodiments, the stable liquid anti-PD1 or anti-PD-L1 antibody are compatible with lyophilization process and the lyophilization process does not impact quality attributes of the antibody.

In any of the above mentioned embodiments, the stable anti-PD1 or anti-PDL1 antibody formulation's osmolality is less than 600 mOsm/kg, preferably less than 350 mOsm/kg.

Another aspect of the invention provides a vial, pre-filled syringe or autoinjector device, or any other suitable device comprising any of the subject formulations described herein. In certain embodiments, the aqueous formulation stored in the vial or pre-filled syringe or autoinjector device contains anti-PD1 or anti-PDL1 antibody, water, mannitol, salt and surfactant.

In any of the above mentioned embodiments, the anti-PD1 antibody is nivolumab, pembrolizumab, cemiplimab or dostarlimab.

In any of the above mentioned embodiment, the anti-PDL1 antibody is atezolizumab, avelumab or durvalumab.

Certain specific aspects and embodiments of the invention are more fully described by reference to the following examples. However, these examples should not be construed as limiting the scope of the invention in any manner.

### EXAMPLES

### Example 1: Buffer Free Anti-PD1 Formulations

As part of experimental design, an anti-PD1 antibody, nivolumab is prepared by recombinant expression of immunoglobulin light and heavy chain genes in a mammalian host cell such as Chinese Hamster Ovary cells. Further, the expressed nivolumab is harvested and the crude harvest is subjected to standard downstream process steps that include purification, filtration and optionally dilution or concentration steps to prepare a buffer free ‘nivolumab formulation, Approximately, 25 mg/ml nivolumab in succinate buffer background was obtained from downstream chromatographic steps. The obtained nivolumab sample was buffer exchanged at least three times with a composition comprising water, post which, 3% mannitol, 2.92 mg/mL NaCl and 0.2 mg/mL polysorbate-80 were added to the sample and concentration of nivolumab was adjusted to 10 mg/ml. This sample is denoted as S1 in Table 1.

Alternatively, another anti-PD1 antibody, pembrolizumab expressed in CHO cells and the expressed antibody has been purified by techniques already known in the art. 35 mg/ml of purified pembrolizumab obtained from downstream chromatographic step, was subjected for buffer exchange step with water. Alternatively, to one of the sample of pembrolizumab in water various excipients were added. Some of the anti-PD1 antibody samples were maintained as such. Composition of all anti-PD1 antibodies samples are given in below Table 1.

Post which, these samples were subjected for accelerated stability studies at 40° C. for one month and various quality attributes of the samples such as change in pH, osmolality; high molecular weight content, monomer content and low molecular weight content using SEC, and charge variants such as acidic variants, basic variants using IEX were measured. Further, these samples were checked for opalescence. Further, the sample was subjected for agitation up to 3 days at 300 RPM using orbital shaker incubator. Results of the study of samples S1 and S2 are given in Table 2 (a), 2(b), 2(c), Table 3 (a), 3(b) and Table 4, whereas the study results of samples P1-P5 are given in Table 5-8.

TABLE 1
Compositions of anti-PD1 antibody formulations
prepared as per Example-1.

| Sample | |
|---|---|
| Name | Composition |
| S1 | 10 mg/ml nivolumab, water, 3% mannitol, 2.92 mg/ml NaCl, |
| | 0.2 mg/ml and polysorbate 80 |
| S2 | 10 mg/ml nivolumab, 10 mM succinate buffer, 3% mannitol, |
| | 2.92 mg/ml NaCl, 0.2 mg/ml and polysorbate 80 |
| P1 | 25 mg/ml pembrolizumab, water |
| P2 | 25 mg/ml pembrolizumab, 10% trehalose, water and |
| | polysorbate-80 |
| P3 | 25 mg/ml pembrolizumab, 4.5% sorbitol, water, 4.5% |
| | trehalose, arginine and polysorbate-80 |
| P4 | 25 mg/ml pembrolizumab, 10% trehalose, 10 mM acetate |
| | buffer and polysorbate-80 |
| P5 | 25 mg/ml pembrolizumab, 4.5% sorbitol, 10 mM acetate |
| | buffer, arginine and polysorbate-80 |

TABLE 2a
SEC data of S1 and S2 samples prepared as per example-1, when
stored at 40° C. for four weeks and agitation for 3 days.

| Formu- | % Monomer content | | | | % HMW species | | | |
|---|---|---|---|---|---|---|---|---|
| lation | | | | Agita- | | | | Agita- |
| details | | T2 W | T4 W | tion_T3D | T0 | T2 W | T4 W | tion_T3D |
| S1 | 99.6 | 99.1 | 98.8 | 99.2 | 0.5 | 0.9 | 1.1 | 0.7 |
| S2 | 99.6 | 99.1 | 98.9 | 99.4 | 0.4 | 0.8 | 1 | 0.6 |

TABLE 2(b)
SEC data of S1 and S2 samples prepares as per example-1,
when stored at 40° C. for four weeks.

| Formulation | % LMW content | | | | |
|---|---|---|---|---|---|
| ID | T0 | T1 W | T2 W | T4 W | Agitation_T3 D |
| S1 | ND | 0.1 | 0.1 | 0.13 | 0.05 |
| S2 | ND | 0.1 | 0.1 | 0.1 | 0.1 |

TABLE 2c
SEC data of S1 sample prepared as per example-1,
when stored at 25° C. for four weeks.

| | % Monomer | | | | | |
|---|---|---|---|---|---|---|
| Formulation | content | | % of HMW | | % of LMW | |
| details | T0 | T4 W | T0 | T4 W | T0 | T4 W |
| S1 | 99.6 | 99.3 | 0.5 | 0.6 | ND | 0.11 |

TABLE 3a
IEX data of S1 and S2 samples prepared as per example-1,
when stored at 40° C. for four weeks.

| | % Main peak content at 40° C. | | | | % Acidic species at 40° C. | | | |
|---|---|---|---|---|---|---|---|---|
| Sample ID | T0 | T1 W | T2 W | T4 W | T0 | T1 W | T2 W | T4 W |
| S1 | 58.4 | 55.8 | 52.0 | 47.1 | 18.4 | 20.7 | 25.1 | 30.1 |
| S2 | 57.2 | 57.3 | 54.4 | 47.4 | 19 | 21.9 | 27 | 33.3 |

TABLE 3(b)
IEX data of S1 and S2 sample prepared as per Example-1,
when stored at 40° C. for four weeks

| Sample | % Basic species at 40° C. | | | | |
|---|---|---|---|---|---|
| ID | T0 | T1 W | T2 W | T4 W | Δ4 W |
| S1 | 23.2 | 23.5 | 22.8 | 22.8 | 0.6 |
| S2 | 23.8 | 20.8 | 18.6 | 19.3 | 4.5 |

In Table 2(a), 2(b), 2(c), 3(a) and 3(b). T0—indicates a value at zero time point; W—indicates—weeks; ND—not detected; D—indicates days: Δ4W-change in the content over a period of four weeks.

TABLE 4
Quality attributes of S1 sample prepared as per Exampe-1

| | Osmolality at 40° C. | | | | pH at 40° C. | | |
|---|---|---|---|---|---|---|---|
| Sample ID | T0 | T1 W | T2 W | T4 W | T0 | T1 W | T2 W |
| S1 | 271 | 303 | 278 | 272 | 6.0 | 5.8 | 5.88 |

TABLE 5
pH measurements of buffer free anti-PD1 antibody formulations
(i.e., P1-P5) prepared as per Example-1.

| | |
|---|---|
| Sample | pH at 40° C. |

| name | T0 | T1 W |
|---|---|---|
| P1 | 6.0 | 6.2 |
| P2 | 5.6 | 5.9 |
| P3 | 5.6 | 5.9 |
| P4 | 5.6 | 5.6 |
| P5 | 5.6 | 5.6 |

TABLE 5
SEC data of P1 to P5 formulations prepared as per
Example-1, when stored at 40° C. for one month.

| | | |
|---|---|---|
| Sample | % HMW content | % Monomer content |

| name | T0 | T1 M | T0 | T1 M |
|---|---|---|---|---|
| P1 | 1.0 | 1.6 | 99.0 | 98.4 |
| P2 | 1.1 | 1.8 | 98.9 | 98.0 |
| P3 | 1.1 | 2.1 | 98.9 | 97.8 |
| P4 | 1.1 | 1.8 | 98.9 | 98.0 |
| P5 | 1.1 | 2.0 | 98.9 | 97.6 |

In Table 4, and 5 T0—represents data at zero time point; W—indicates weeks; M—indicates months

TABLE 6
IEX data of P1 to P5 formulations prepared as per
Example-1, when stored at 40° C. for one weak.

| | % Acidic | | % Main peak | | | | |
|---|---|---|---|---|---|---|---|
| Sample | variants | | content | | % Basic variants content | | |
| name | T0 | T1 W | T0 | T1 W | T0 | T1 W | Δ4 W |
| P1 | 7.9 | 15.3 | 74.2 | 68.5 | 17.9 | 16.2 | 0.7 |
| P2 | 9.5 | 12.5 | 62.5 | 60.3 | 28.0 | 27.2 | 0.8 |
| P3 | 9.7 | 12.5 | 63.3 | 60.1 | 27.0 | 27.3 | 0.3 |
| P4 | 9.5 | 12.2 | 63.5 | 60.6 | 27.0 | 27.2 | 0.2 |
| P5 | 8.6 | 12.6 | 59.0 | 60.7 | 32.0 | 26.7 | 5.3 |

T0—represents data at zero time point; M—indicates months: Δ4W—change in the content over a period of four weeks.

TABLE 8
Opalescence of P1 to P5 samples prepared as per example 1.

| Sample | Opalescence at 40° C. | | | |
|---|---|---|---|---|
| Name | 0 W | 1 W | 2 W | 4 W |

| | | |
|---|---|---|
| P1 | ROS-I-II and no visible | ROS-I-II and no visible |
| | particles | particles |
| P2 | ROS-I-II and no visible | ROS-III-IV and no visible |
| | particles | particles |
| P3 | ROS-II to III and no visible | ROS-III-IV and small fibrous |
| | particles | particles |
| P4 | ROS-II to III and no visible | ROS-II and III |
| | particles | |
| P5 | ROS-I and no visible particles | ROS-III |

### Example 2: High Concentration Anti-PD1 Antibody in Buffer Free Formulation

10 mg/ml of nivolumab in succinate buffer, comprising 60 mg/ml trehalose, methionine, 2.92 mg/ml sodium chloride, 0.008 mg/ml DTPA and 0.2 mg/ml polysorbate-80 was further concentrated up to 120 mg/ml by ultrafiltration. This high concentration nivolumab sample in buffer was buffer exchanged into water. Post which, the sample was subjected for stress stability condition at 40° C. for one week and measured for HMW species, monomer content and low molecular weight species using SEC. Further, acidic variants and main peak contents of the samples were measured using IEX chromatography and viscosity of the sample was measured using viscometer. Results of the study are given below in Table 9.

TABLE 9
Quality attributes of the formulations at 40° C.

| | % HMW | | % of | | % of acidic | | % of main | |
|---|---|---|---|---|---|---|---|---|
| Sample | content | | monomer | | variants | | peak content | |
| composition | T0 | T1 W | T0 | T1 W | T0 | T1 W | T0 | T1 W |
| 115 mg/ml | 1.1 | 2.1 | 98.9 | 97.8 | 15.2 | 10.3 | 60.6 | 58.7 |
| nivolumab, | | | | | | | | |
| 60 mg/ml | | | | | | | | |
| trehalose, | | | | | | | | |
| 10 mM | | | | | | | | |
| methionine, | | | | | | | | |
| 0.008 mg/ml | | | | | | | | |
| DTPA, | | | | | | | | |
| 0.2 mg/ml | | | | | | | | |
| polysorbate | | | | | | | | |

1. A liquid buffer free formulation of an anti-PD1 or an anti-PD-L1 antibody, comprising anti-PD1 or anti-PD-L1 antibody and water, wherein the pH of the formulation is maintained at a pH of about 5.0 to about 6.0 without any buffering agent.

2. The buffer free formulation according to claim 1, wherein the antibody concentration is from 10 mg/ml to 200 mg/ml.

3. The buffer free formulation according to claim 1, further comprises sugar or polyol, salt and/or surfactant.

4. A method of stabilizing an anti-PD1 or an anti-PD-L1 antibody in a solution, at a pH of about 5.0 to about 6.0 without a buffering agent, the method comprising;
a) expressing and purifying an anti-PD1 or an anti-PDL1 antibody to obtain the antibody composition,
b) subjecting the purified antibody composition to a diafiltration step using water as a diafiltration medium,
c) obtaining the antibody composition in water to obtain anti-PD1/anti-PDL1 antibody solution.

5. The method according to claim 4, further comprises addition of addition of polyol and/or salt and/or surfactant to the antibody composition obtained from step c).

6. (canceled)

7. The buffer free formulation according to claim 1, wherein the antibody formulated resists any change in basic variants content over time, as compared to the basic variants content of the antibody formulated in buffer.

8. The buffer free formulation according to claim 7, wherein the change in the basic variants content of the antibody formulated in water is less than 1%.

9. The buffer free formulation according to claim 1, wherein the anti-PD1 antibody is nivolumab or pembrolizumab.

10. The buffer free formulation according to claim 1, wherein the anti-PD1 antibody is atezolizumab or durvalumab.

11. The method according to claim 4, wherein the antibody formulated resists any change in basic variants content over time, as compared to the basic variants content of the antibody formulated in buffer.

12. The method according to claim 4, wherein the change in the basic variants content of the antibody formulated in water is less than 1%.

13. The method according to claim 4, wherein the anti-PD1 antibody is nivolumab or pembrolizumab.

14. The method according to claim 4, wherein the anti-PD1 antibody is atezolizumab or durvalumab.
